(19) United States
(12) Patent Application Publication
(10) Pub. No.: US 2012/0078227 A1
(43) Pub. Date: Mar. 29, 2012

(54) Drug Coated Balloon Composition with High Drug Transfer to Vessel

(75) Inventor: Steve Kangas, Woodbury, MN (US)

(73) Assignee: BOSTON SCIENTIFIC SCIMED, INC., Maple Grove, MN (US)

(21) Appl. No.: 13/217,528

(22) Filed: Aug. 25, 2011

Related U.S. Application Data
(60) Provisional application No. 61/385,849, filed on Sep. 23, 2010.

(51) Int. Cl. A61L 29/16; B05D 3/10

(52) U.S. Cl. 604/509; 604/103.02; 427/2.3

(57) ABSTRACT

Drug delivery balloons are configured with a carrier film of biodegradable polymer. The carrier film includes a drug or has a drug carried thereon. The balloons utilize a film layering system that is designed to separate the carrier film substantially intact from the balloon when expanded, so that the carrier film and the drug are left in place at the tissue site.

### CROSS REFERENCE TO RELATED APPLICATIONS

This application claims priority to U.S. provisional application 61/385,849, filed Sep. 23, 2010, which is incorporated herein by reference in its entirety.

### BACKGROUND OF THE INVENTION

Percutaneous intravascular procedures have been developed for treating atherosclerotic disease in a patient's vasculature. The most successful of these treatments is percutaneous transluminal angioplasty (PTA). PTA employs a catheter having an expansible distal end, usually in the form of an inflatable balloon, to dilate a stenotic region in the vasculature to restore adequate blood flow beyond the stenosis.

Sometimes following an initially successful angioplasty or other primary treatment restenosis occurs within weeks or months of the primary procedure. Restenosis results at least in part from smooth muscle cell proliferation in response to the injury caused by the primary treatment. This cell proliferation is referred to as “hyperplasia.” Blood vessels in which significant restenosis occurs will typically require further treatment.

A number of strategies have been proposed to treat hyperplasia and reduce restenosis. Previously proposed strategies include prolonged balloon inflation, treatment of the blood vessel with a heated balloon, treatment of the blood vessel with radiation, the administration of anti-thrombotic drugs following the primary treatment, stenting of the region following the primary treatment, the use of drug-eluting stents, use of drug delivery balloons, cutting balloons, cryotherapy systems and the like.

Drug delivery balloons that deliver drug to an internal site upon expansion are known. Some involve perfusion of a drug composition through the balloon wall or from a spongy layer on the balloon wall. Others involve delivery of solid particulate drug, often carried in a polymer or other excipient to the site.

Delivery of drug from the surface during expansion provides benefits of pushing the drug into the specific tissue to be effected and is especially suited for delivering drugs that prevent restenosis during a dilation of a stenotic lesion. However the delivery technique still suffers from a fundamental conflict between the contradictory needs to deliver an effective dose at the treatment site and to keep the drug adhering to the balloon as it is being manipulated to that site. Techniques to improve drug adhesion, such as formulation with polymers or other excipients or application of protective layers, make it more difficult to effectively deliver an effective dose when the balloon is inflated. Conversely if the drug is applied to the balloon unformulated, or is formulated with a highly soluble excipient, for instance contrast agents such as iopromide, or sugars such as sucrose or mannitol, undesirably high losses and dosage variation can result. In early commercial balloons of this type as much as 85% of the drug has been carried away from the drug site in the form of particulates of various sizes and transfer efficiency is only about 2-10%.

Known drug delivery balloons include paclitaxel coated balloons. In some cases paclitaxel has been applied directly to the balloon or to a coating placed on the balloon. In other cases paclitaxel has been formulated with an excipient that may be polymer, a contrast agent, a surface active agent, or other small molecules that facilitate adhesion to the balloon and/or release from the balloon upon expansion. The formulations have typically been applied from solution, and may be applied to the entire balloon or to a folded balloon, either by spraying, immersion or by pipette along the fold lines. However the commercial balloons do not yet provide for delivery of predictable amounts of the drug to the tissue at the delivery site nor do they provide for a predictable therapeutic drug tissue level over an extended time period.

Earlier investigations of paclitaxel coated balloons by the applicant have shown that it is desirable to control the morphology of the drug on the balloon, that with paclitaxel coated balloons paclitaxel dihydrate paclitaxel crystalline form facilitates longer tissue residence time, that the formation of crystalline paclitaxel dihydrate can be controlled by use of vapor annealing of the balloon, and that temperature change at the delivery site can be used to trigger a change in the bonding properties of a drug or drug-containing composition to the balloon.

There is an ongoing need for improved drug delivery balloon devices, systems and methods.

### SUMMARY OF THE INVENTION

The invention provides novel techniques and structures to solve problems of balloon structures such as drug delivery coatings.

In at least some embodiments the invention pertains to drug delivery balloons that are configured with a carrier film of biodegradable polymer. The carrier film includes a drug or has a drug carried thereon. The balloons utilize a film layering system that is designed to separate the carrier film substantially intact from the balloon when expanded, so that the carrier film and the drug are left in place at the tissue site.

In some embodiments the carrier film of biodegradable polymer is a drug containing matrix material and is transferred substantially intact from the balloon to tissue upon expansion. In other embodiments the carrier film of biodegradable polymer is provided intermediate a drug-containing layer and the balloon and both the biodegradable polymer layer and the drug are transferred substantially intact to the tissue.

One particular aspect of the invention pertains to a drug delivery balloon comprising:

a balloon wall, and

a carrier film of biodegradable polymer that
includes a drug therein, or
has a drug carried thereon, and
   said carrier film is sufficiently non-adherent to the balloon wall that it can separate substantially entirely from the balloon wall when the balloon is expanded at a delivery site and remain in place at the tissue site.

Yet another aspect of the invention pertains to method of delivering a drug to a treatment site in the body comprising

providing a medical device comprising a drug delivery balloon of the invention,

advancing the balloon to the treatment site,

inflating the balloon,

deflating the balloon to separate said carrier film with said drug from the balloon wall, and

withdrawing the balloon leaving the carrier film with said drug in place at the treatment site.

Another particular aspect of the invention pertains to a process for producing a medical device balloon comprising the steps of

providing a balloon having a balloon wall,

applying a layer of an extractable material to the balloon wall,

applying a biodegradable carrier film on the extractable layer, and

extracting the extractable layer to leave an unbonded layer of carrier film on the balloon.

Still another particular aspect of the invention pertains to a process for producing a medical device balloon comprising the steps of

providing a balloon having a balloon wall,

applying a biodegradable ionically crosslinkable polymer film to the balloon wall, and

ionically crosslinking the ionically crosslinkable polymer film.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIGS. 1-5 are schematic cross-sectional depictions illustrating a method of preparing a drug delivery balloon of the invention and use of the balloon to deliver a drug.

FIGS. 6-8 are schematic cross-sectional depictions illustrating another method of preparing a drug delivery balloon of the invention and use of the balloon to deliver a drug.

FIG. 9a is a photographic image of a balloon of the invention, prepared as described in Example 1, before deployment in a transparent polymer tube. FIG. 9b is a photographic image of the tube after deployment.

FIG. 10a is a photographic image of a tube in which a drug has been delivered using a balloon of the invention as described in Example 2. FIG. 10b shows the balloon after delivery.

FIGS. 11a and 11b are photographic images, respectively, of tube and balloon after delivery using a control balloon as described in Example 2

### DETAILED DESCRIPTION OF PREFERRED EMBODIMENTS

All published documents, including all US patent documents, mentioned anywhere in this application are hereby expressly incorporated herein by reference in their entirety. Any copending patent applications, mentioned anywhere in this application are also hereby expressly incorporated herein by reference in their entirety.

Drug delivery balloon systems are known and have been described in at least the following documents:
U.S. Pat. No. 5,102,402, Dror et al (Medtronic, Inc.);
U.S. Pat. No. 5,370,614, Amundson et al, (Medtronic, Inc.);
WO 9916500, Medtronic,
WO 2009066330, Medtronic
U.S. Pat. No. 5,954,706, Sahatjian (Boston Scientific Corp);
WO 00/32267, SciMed Life Systems; St Elizabeth's Medical Center (Palasis et al); WO 00/45744, SciMed Life Systems (Yang et al);
R. Charles, et al, “Ceramide-Coated Balloon Catheters Limit Neointimal Hyperplasia After Stretch Injury in Cartoid Arteries,” Circ. Res. 2000; 87; 282-288;
U.S. Pat. No. 6,306,166, Barry et al, (SciMed Life Systems, Inc.);
US 2004/0073284, Bates et al (Cook, Inc; MED Inst, Inc.);
US 2006/0020243, Speck;
WO 2008/003298 Hemoteq AG, (Hoffman et al);
WO 2008/086794 Hemoteq AG, (Hoffman et al);
US 2008/0118544, Wang;
US 2008/0255509, Wang (Lutonix); and
US 2008/0255510, Wang (Lutonix);
US 2010/0055294, Wang, (Lutonix);
US 2010/076542, Orlowski, (Eurocore);
US 2010/0145266, Orlowski, (Eurocore);
   and in the following U.S. patent applications:
Ser. No. 12/765,522 filed Apr. 22, 2010, claiming benefit of U.S. provisional application 61/172,629, filed Apr. 24, 2009, entitled “Use of Drug Polymorphs to Achieve Controlled Drug Delivery From a Coated Medical Device;”
Ser. No. 12/815,138, filed Jun. 14, 2010, claiming benefit of U.S. provisional application 61/224,723, filed Jul. 10, 2009, entitled “Use of Nanocrystals for a Drug Delivery Balloon; Ser. No. 12/815,138, filed Jun. 14, 2010, 61/271,167, filed Jul. 17, 2009, entitled “Nucleation of Drug Delivery Balloons to Provide Improved Crystal Size and Density;” and
U.S. provisional application 61/291,616, filed Dec. 31, 2010, entitled “Cryo Activated Drug Delivery Cutting Balloon.”

The present invention contemplates methods and structures that result in transfer of a substantially intact biodegradable carrier film to a tissue layer.

Compared to the extremely low transfer efficiency of current balloon products, the carrier films transfer substantially entirely from the balloon to the treatment site. The drugs can be incorporated into or adhered to the carrier film so as to achieve a comparable (in some cases substantially 100%) transfer efficiency.

The carrier film is folded with the balloon during tracking and so remains intact due to the physical folding of the balloon. Upon deployment, the carrier film releases from the balloon, with the drug, and adheres to the vessel. After deployment the polymer degrades leaving the drug on the vessel.

In some embodiments the drug is provided as a layer on the biodegradable carrier film layer. An advantage to including the drug on the surface of the carrier film is that one can formulate with a fast degrading/dissolving polymer, but as the polymer degrades the drug remains at the treatment site and less is released systemically.

For purposes of the invention the term drug includes both therapeutic agents and diagnostic agents. Non-limiting examples of drugs that may be employed include anti-restenosis agents, antiproliferative agents, antibiotic agents, antimitotic agents, antiplatelet agents, alkylating agents, platinum coordination complexes, hormones, anticoagulants, fibrinolytic agents, antimigratory agents, antisecretory agents, anti-inflammatory agents, indole acetic acids, indene acetic acids, immunosuppressive agents, angiogenic agents, angiotensen receptor blockers, nitric oxide donors, anti-sense oligonucleotides, cell cycle inhibitors, mTOR inhibitors, growth factor receptor signal inhibitors, transduction kinase inhibitors, retenoids, cyclin/CDK inhibitors, HMG co-enzyme reductase inhibitors, protease inhibitors, viral gene vectors, macrophages, monoclonal antibodies, x-ray contrast agents, MRI contrast agents, ultrasound contrast agents, chromogenic dyes, fluorescent dyes, and luminescent dyes.

In some embodiments the drug is a lipophilic substantially water insoluble drug, such as paclitaxel, rapamycin (also known as sirolimus), everolimus, zotarolimus, biolimus A9, dexamethasone, tranilast or another drug that inhibits restenosis. Other drugs that may be suitable are described in the documents incorporated elsewhere herein. Mixtures of drugs, for instance two or more of paclitaxel, rapamycin, everolimus, zotarolimus, biolimus A9, dexamethasone and/or tranilast may be employed.

Further examples of drugs include estrogen or estrogen derivatives; heparin or another thrombin inhibitor, hirudin, hirulog, argatroban, D-phenylalanyl-L-poly-L-arginyl chloromethyl ketone or another antithrombogenic agent, or mixtures thereof, urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; an antimicrobial agent or antibiotic; aspirin, ticlopdine or another antiplatelet agent; colchicine or another antimitotic, or another microtubule inhibitor; cytochalasin or another actin inhibitor; a remodelling inhibitor; deoxyribonucleic acid, an antisense nucleotide or another agent for molecular genetic intervention; GP IIb/IIIa, GP Ib-IX or another inhibitor or surface glycoprotein receptor; methotrexate or another antimetabolite or antiproliferative agent; an anticancer chemotherapeutic agent; dexamethasone, dexamethasone sodium phosphate, dexamethasone acetate or another dexamethasone derivative, or another anti-inflammatory steroid; dopamine, bromocriptine mesylate, pergolide mesylate or another dopamine agonist; a radiotherapeutic agent; iodine-containing compounds, barium-containing compounds, gold, tantalum, platinum, tungsten or another heavy metal functioning as a radiopaque agent; a peptide, a protein, an enzyme, an extracellular matrix component, a cellular component or another biologic agent; captopril, enalapril or another angiotensin converting enzyme (ACE) inhibitor; ascorbic acid, alphatocopherol, superoxide dismutase, deferoxyamine, a 21-aminosteroid (lasaroid) or another free radical scavenger, iron chelator or antioxidant; angiopeptin; a radiolabelled form of any of the foregoing; or a mixture of any of these.

The drug may be one that has polymorph forms, i.e. at least two characterizable morphologies that have different solubilities, or crystal forms. In some embodiments the different morphological forms have characteristics that affect tissue uptake of the drug at the delivery site. Drugs such as paclitaxel have more than one such morphological form. These have different solubilities and dissolution rates in body fluids, including blood. For some embodiments the drug is provided in a specific polymorph form(s) or distribution of such forms to facilitate a particular therapeutic objective. In some cases the drug also is provided in a particulate size profile that facilitates uptake by the adjacent tissue rather than dissolving into the blood stream and some fraction taken up by the vessel (the therapeutic dose). Very small particles, <1 μm, can be taken up directly into the arterial tissue. Some of the drug that diffuses into the vessel wall binds to and stabilizes the cell microtubules, thereby affecting the restenotic cascade after injury of the artery.

In exemplary embodiments a drug coating on or in the carrier film comprises dose density of between 0.25 μg mm² and 5 μg/mm² of a drug, for instance paclitaxel, rapamycin, everolimus, zotarolimus, biolimus A9, dexamethasone and/or tranilast.

In some embodiments of a paclitaxel containing drug coating, the fraction of the paclitaxel in the coating that is amorphous is from 0-25%, for instance about 1% to about 5%, based on total paclitaxel weight. In some embodiments the fraction of the paclitaxel in the coating that is anhydrous from 0% to about 99%, for instance 5-95%, about 10%, about 15%, about 20%, about 25%, about 30%, about 40%, about 50%, about 60%, about 70%, about 70%, or about 80%, based on total paclitaxel weight. In some embodiments the fraction the paclitaxel in the coating that is dihydrate crystalline is from 1% to 100%, for instance 1-99%, 5-95%, about 10%, about 15%, about 20%, about 25%, about 30%, about 35%, about 40%, about 45%, about 50%, about 55%, about 60%, about 65%, about 70%, about 75%, about 80%, about 85%, about 88%, about 89%, about 90%, about 91%, about 92%, about 93%, about 94%, about 95%, about 96%, about 97%, about 98%, or about 99%, based on total paclitaxel weight.

In some embodiments the drug may be a solid crystalline form that includes organic solvent molecules such as dimethylsulfoxide (DMSO), N,N′-dimethylformamide (DMF), N,N′-dimethylacetamide (DMAC), N-methyl-2-pyrrolidone (NMPO), 1,3-dimethyl-2-imidazolidinone (DMEU), 1,3-dimethyl-3,4,5,6-tetrahydro-2-(1H)-pyrimidinone (DMPU), and acetonitrile and mixtures thereof, with or without water molecules in the crystal structure. Examples of such crystalline forms include the paclitaxel solvates described in U.S. Pat. No. 6,858,644. In some embodiments the drug may be dried dispersant-containing drug particle such as described in U.S. Pat. No. 6,780,324.

In some embodiments the drug in a drug coating is in a particulate form that has a particle size in the range of 0.01-20.0 μm (10-20000 nm). Multi-modal ranges, prepared, e.g. by mixing two or more sets of different narrow size range may be used in some cases to provide a desired bioavailability profile over time. For example 50% of the crystals can be of 1000 nm mean size and the other 50% could be 300 nm mean size. These embodiments enable a tailoring of the drug persistence in the vessel wall. The smaller crystals will more readily dissolve and enter the tissue for immediate effect and larger crystals will dissolve at a much slower rate enabling longer drug persistence. In some embodiments the drug particles may take the form of microparticles (i.e. the drug particle does not include an encapsulant enclosing the drug), which are in turn mixed with a polymeric carrier to form a drug coating. Paclitaxel crystalline dihydrate is exemplary of a suitable particulate drug that may be usefully be utilized with such multi-modal size distributions.

According to the invention the drug or drugs are carried on or are included in a biodegradable carrier film. The carrier film should have no adhesion to the balloon or at least sufficiently low adhesion that upon balloon expansion and retraction the film will separate substantially intact from the balloon. For tracking to the delivery site balloon adhesion is not needed because the folding of the balloon mechanically restricts the film from being dislodged from the balloon. The carrier film also needs to have sufficient cohesive strength to remain intact during tracking to the delivery site and upon delivery.

Embodiments of the invention can utilize biodegradable polymer that are synthetic or natural in origin and natural polymers may be modified in known ways that increase their suitability as carrier films or their degradation rate while retaining biodegradability.

Biodegradable polymers include polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, poly-α-hydroxy acids, trimethylene carbonate, poly-β-hydroxy acids, polyorganophosphazines, polyesteramides, polyethylene oxide, polyester-ethers, polyphosphoester, polyphosphoester urethane, cyanoacrylates, poly(trimethylene carbonate), poly(iminocarbonate), polyalkylene oxalates, polyvinylpyrolidone, polyvinyl alcohol, poly-N-(2-hydroxypropyl)-methacrylamide, polyglycols, aliphatic polyesters, poly(orthoesters), poly(ester-amides), polyanhydrides, polysaccharides, and proteins such as gelatin. Specific examples include polyhydroxyalkanoates (PHA), polyhydroxybutyrate compounds, and co-polymers and mixtures thereof, poly(glycerol-sebacate), polypeptides, poly-α-hydroxy acid, such as polylactic acid (PLA). PLA can be a mixture of enantiomers typically referred to as poly-D,L-lactic acid. Alternatively, the biodegradable material is poly-L(+)-lactic acid (PLLA) or poly-D(−)-lactic acid (PDLA), which differ from each other in their rate of biodegradation. PLLA is semicrystalline. In contrast, PDLA is amorphous, which can promote the homogeneous dispersion of an active species. Other examples include polyglycolide (PGA), copolymers of lactide and glycolide (PLGA), polydioxanone, polygluconate, polylactic acid-polyethylene oxide copolymers, alginates, hyaluronic acid. Mixtures of such materials may be used.

In some embodiments the biodegradable carrier film polymer is a biodegradable polymer that would normally bond to the balloon after application from solution or dispersion making separation impractical. An example of a polymer that can will normally bond to a balloon are lactate polymers and copolymers such as PLGA. In such cases a release layer may be interposed between the carrier film and the balloon wall. In some embodiments a release layer may be a material that can be extracted after the carrier film is applied. Extracting the material leaves the film surrounding the balloon but not adherent thereto. In other embodiments it may be possible to use a lubricant as a release layer. However applying the carrier film coating to a lubricant layer can be difficult and may not be as practical as use of an extractable material.

The PLGA polymers may be fairly rigid when dry but typically they plasticize rapidly in contact with water and so have sufficient cohesive strength in water to be readily folded with the balloon and unfolded at the treatment site to unfold and conform to a vessel wall when the balloon is expanded. PLGA polymers also have well characterized degradation profiles that allow for tailoring of the film degradation time. To provide for release of a PLGA carrier film layer from the balloon, the balloon can be first coated with a material that can be extracted through the PLGA film. A suitable material is a low molecular weight polyvinyl pyrrolidone, for instance a PVP having a number average molecular weight of about 50,000 or less, for instance about 20,000 or less, or about 5,000 to about 10,000. Low molecular weight PVP can be applied from solvent or aqueous solution, overcoated with a PLGA film and extracted by soaking the overcoated balloon in water for a short time, for instance 5 minutes to several hours at ambient temperature. Similar techniques can be used with other polymers that would otherwise bind too strongly to the balloon to prevent coherent film release.

In other embodiments crosslinked polysaccharides may be utilized as a biodegradable carrier film polymer. In some cases a polysaccharide or other natural or synthetic polymer may be chemically crosslinked, for instance with glutaraldehyde or another compound having at least two aldehyde groups to provide a biodegradable carrier film. Chemically crosslinked biodegradable films may also require use of a release material between the balloon and the carrier film layer.

In some embodiments the crosslinking may be ionic. In such case the crosslinked films will often have low enough balloon adhesion to avoid the need for an extractable layer. Ionic crosslinking can both increase the cohesive film strength of the material and at the same time reduce the adhesion of the carrier film to the balloon (as compared to the uncrosslinked polymers). For instance a polysaccharide which has acid functional groups thereon may be used. Acid functionality may be provided by carboxylate or sulfate groups, or both. Alginates are exemplary ionically crosslinkable polysaccharides. Glycosaminoglycans, for instance hyaluronic acid, xanthan gum, carrageenan, tragacanth, gellan gum and pectins are examples of suitable acid functional polysaccharides. Crosslinking is conveniently provided with a biocompatible polyvalent cation such as calcium, magnesium or iron. In some cases a polymer with multiple cationic groups may be utilized.

In some embodiments the biodegradable carrier film is formulated to substantially degrade or dissolve rapidly, for instance within a few days or less, for instance from about 5 minutes to about 24 hours after delivery. In other cases the biodegradable carrier film may be formulated to degrade over a longer period of several days, weeks or months, for instance from about 5 days to about 6 months. During this time if the drug is adjacent to the carrier film it will be held at the tissue site until taken up or until the film degrades. If the carrier film is a drug matrix the drug will be made available at the site as the film degrades. In some cases it may be desirable to utilize both forms of delivery either to provide a desired initial and extended release profile of a single drug or to provide different drugs with independent release profiles.

The carrier film may be formulated as a drug matrix or be placed intermediate between the drug containing layer and the balloon. In both cases, delivery of the carrier film substantially intact also delivers the drug to the treatment site.

The thickness of the carrier film will influence the degradation time of the carrier film in the body, and in some cases the film thickness will be selected to provide a particular degradation time. In general it is desirable to provide a carrier film that has a thickness of less than 10 μm, for instance from 0.1 μm to 5 μm, from 0.5 to 2 μm, or less than 1 μm.

Optionally a drug layer may also include an excipient or additive including for instance, citrate esters, such as tributyl citrate, triethyl citrate, acetyltributyl citrate, and acetyltriethyl citrate; polyols, such as glycerin, polyglycerin, sorbitol, polyethylene glycol and polypropylene glycol; starches; vegetable oils; fats; glucose or sucrose ethers and esters; polyethylene glycol ethers and esters; low toxicity phthalates; alkyl phosphate esters; dialkylether diesters; tricarboxylic esters; epoxidized oils; epoxidized esters; polyesters; polyglycol diesters; aliphatic diesters, for instance dibutyl sebacate; alkylether monoesters; dicarboxylic esters; lecithin; and/or combinations thereof. Numerous other excipients and additive compounds, are described in one or more of the documents incorporated herein by reference. In some cases the excipient may increase the adhesion of the drug to the carrier film layer. An excipient is not needed to induce release from the balloon, but it can be useful for other purposes. For instance, to promote adhesion of the drug to the carrier film layer or to facilitate uptake of the drug into the tissue at the site. In some embodiments a drug layer may include a polymer different from the biodegradable film layer as an excipient.

In some embodiments multiple drugs are provided which may be in mixture or physically separated, e.g. as discrete particles or in separate layers. For instance one drug may be provided in the biodegradable film polymer layer and a second over it. Alternatively, multiple drugs may be applied to a film polymer layer from a single solution or emulsion. In another example multiple drugs are applied to a film polymer layer from two different solutions, concurrently (e.g. by spraying) or successively (spraying, dipping, wiping or the like), to give one or several drug containing layers.

In some embodiments the drug is delivered in a formulation that provides for extended release into adjacent tissue. While this possibility has been recognized previously for drug delivery balloons, the problems of the inefficiency of delivery have significantly limited the design options for extended release formulations on drug delivery balloons.

The balloons, may be elastic and/or inelastic balloons, and may be formed of material such as polyamides (for example, nylon 12 or DURETHAN® available from Bayer or CRISTAMID® available from Elf Atochem), polyethylene terephathalate (PET), polyurethane, latex, silicone, polyethylene (PE) (for example, Marlex® high-density polyethylene, Marlex® low-density polyethylene, and a linear low density polyethylene such as REXELL®), polypropylene (PP), polyetherimide (PEI), polytetrafluoroethylene (PTFE), ethylene tetrafluoroethylene (ETFE), fluorinated ethylene propylene (FEP), polyoxymethylene (POM), polybutylene terephthalate (PBT), polyether-block-ester (for example, a polyether-block-ester elastomer such as ARNITEL® available from DSM Engineering Plastics or a polyester elastomer such as HYTREL® available from DuPont), polyvinylchloride (PVC), polyether-block-amide (PEBA, for example, available under the trade name PEBAX®), polyetheretherketone (PEEK), polyimide (PI), polyphenylene sulfide (PPS), polyphenylene oxide (PPO), poly(ethylene naphthalenedicarboxylate) (PEN), polysulfone, perfluoro(propyl vinyl ether) (PFA), or mixtures, combinations, copolymers thereof, and the like.

In some embodiments the balloon wall is formed of one or more layers of Pebax® polymers, suitably Pebax® 6333, Pebax® 7033, Pebax® 7233; nylon polymers, for instance nylon 11 or nylon 12; or a mixture thereof.

The balloon will typically have a length of at least 1 cm, preferably being in a range from about 1.5 cm to 20 cm, and may have diameters in a range from 1.5 mm to about 20 mm, for instance 1.5 to 5 mm

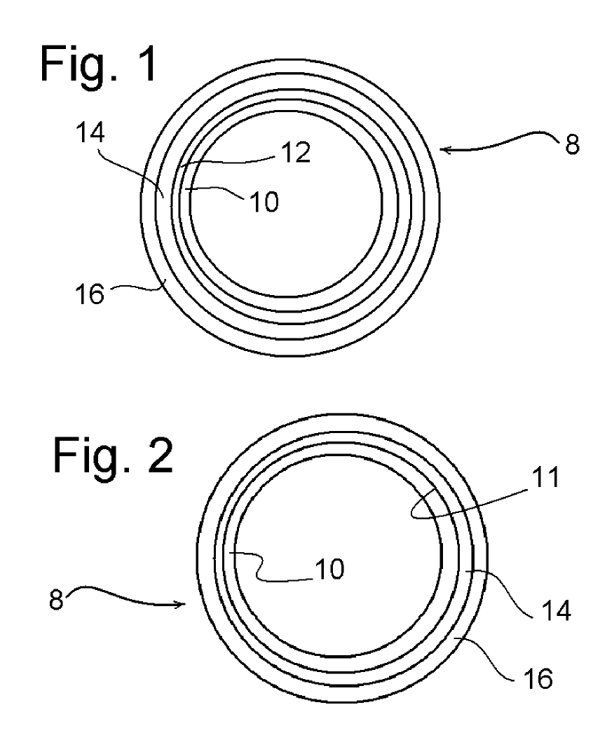

Referring to FIG. 1, there is shown schematically a cross sectional view of a balloon 8 having a balloon wall 10, a dissolvable release layer 12, a degradable carrier film layer 14 and a drug layer 16 arranged concentrically.

Treatment of the balloon 8 with water after application of the drug layer will remove at least some of the dissolvable layer 12 to loosen the layer 14 from adhering to the balloon, as shown in FIG. 2. Drug layer 16 retains adhered to the carrier film layer 14 so that the balloon can be folded, sterilized and deployed. In an alternative embodiment, not shown, the layer 12 may be extracted from beneath the layer 14 before the drug layer 16 is formed.

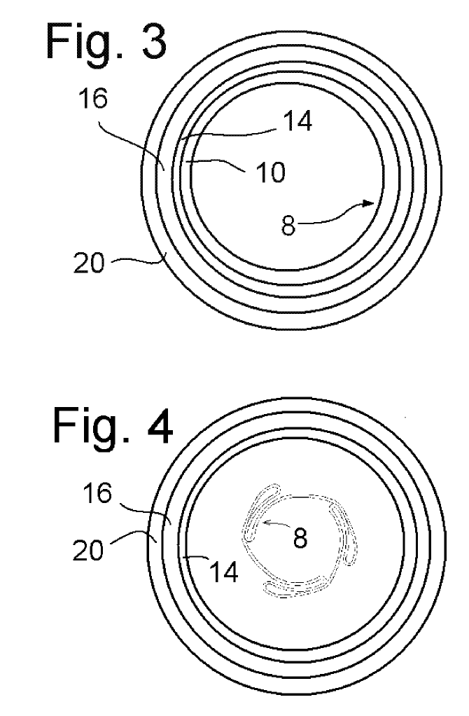

FIG. 3 shows the balloon 8, after extraction of the release layer 12, expanded in a vessel 20. The layers 14, 16 are pressed against the vessel.

FIG. 4 shows the balloon 8 in phantom deflated and withdrawn from the film, leaving the layers 14, 16 in place in the vessel 20.

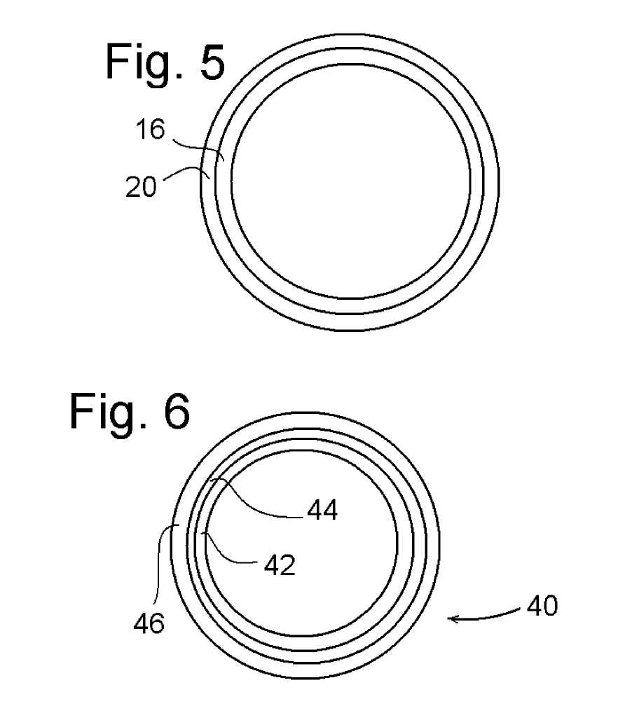

FIG. 5 shows a point in time after deployment. Layer 14 degrades in the body leaving the drug layer 16 in place on vessel 20. In other embodiments the layer 14 may be configured to degrade slow enough that the drug is taken up by the tissue of vessel 20 before layer 14 has fully degraded.

In still other embodiments, not shown, the layer 16 is eliminated and instead the layer 14 is configured to include both a degradable polymer and the drug to be delivered. A dissolvable layer 12 is initially provided and it is again removed in the course of manufacturing. Deployment leaves the drug containing layer in place in the vessel and degradation of the polymer makes the drug available to the tissue over an extended period of time.

FIG. 6 shows an alternate embodiment of the invention. Balloon 40 has a balloon wall 42 coated with an ionically crosslinked biodegradable carrier film material shown as layer 44. The ionically crosslinked biodegradable carrier film material is one that has little or no adhesion to balloon 40 after it is crosslinked. A drug layer 46 is applied to the ionically crosslinked biodegradable carrier film layer 44.

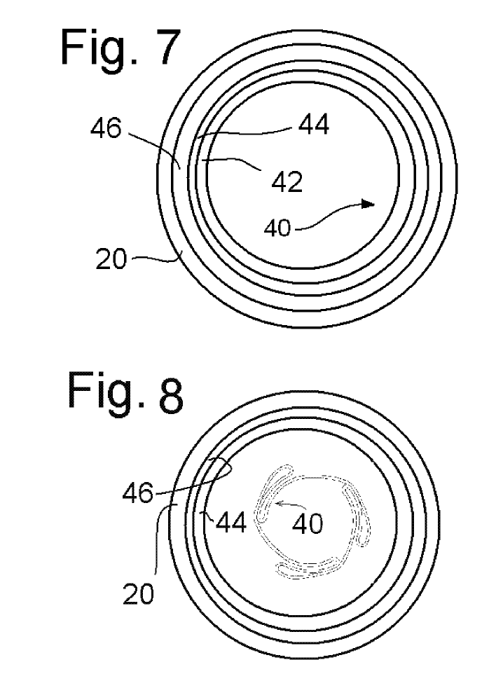

FIG. 7 shows the balloon 40 deployed in a vessel 20.

Pressing the carrier film and drug layers against the wall of the vessel 20, and then deflating the balloon releases both layers from the balloon when the balloon 40 is deflated, as depicted in FIG. 8.

Depending on the particular drug the crosslinked carrier film may degrade before or after the drug has been taken up into the tissue of the vessel 20. In some embodiments the drug is one or more of paclitaxel, rapamycin, everolimus, zotarolimus, biolimus A9, dexamethasone and/or tranilast and the ionically crosslinked biodegradable carrier film material degrades faster than the uptake of the drug.

In further alternate embodiments, not shown, the drug and crosslinked carrier film polymer are provided in a single layer, rather than the two layers 44 and 46.

The devices of the present invention, may be deployed in vascular passageways, including veins and arteries, for instance coronary arteries, renal arteries, peripheral arteries including illiac arteries, arteries of the neck and cerebral arteries, and may also be advantageously employed in other body structures, including but not limited to arteries, veins, biliary ducts, urethras, fallopian tubes, bronchial tubes, the trachea, the esophagus and the prostate.

The invention is illustrated by the following non-limiting examples.

### Example 1

### PLGA Carrier Film

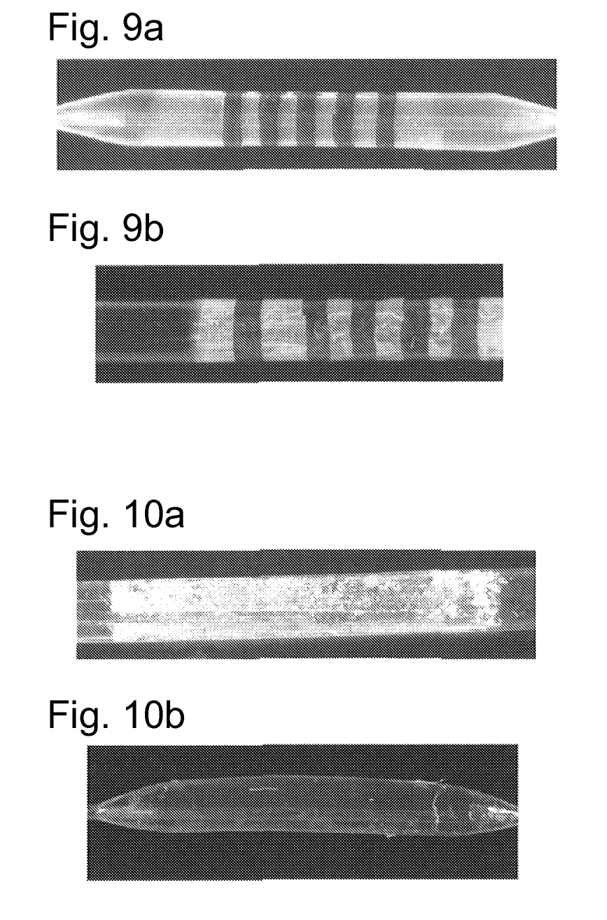

A 3×15 mm Liberte® balloon was dip-coated (0.25 inches/sec with a ˜1 sec hold time) in a 40% solution of PVP (10K MW) in IPA and dried. The PVP coated balloon was then dip coated (0.25 inches/sec, with a ˜1 sec hold time) in a 20% solids solution of 50/50 PLGA (4.5 A) in THF and dried. The coated balloon was then immersed in water or about 30 minutes to effect dissolution of the PVP layer. The balloon was then dried and folded. Prior to folding, the PLGA coating was marked with a Sharpie™ marker to mark the PLGA coating to aid in visualization. The Sharpie™ ink provides a visual proxy for a hydrophobic drug. The folded balloon was then inserted into a synthetic artery consisting of a Tecophilic® polyurethane tube (2.75 mm diameter) in water at 37° C. The balloon was held in the tube for 1 min and then inflated to 16 atm for 1 min. Vacuum was then pulled and the balloon was removed from the tube. FIG. 9a shows an optical image of the balloon before deployment. FIG. 9b shows the polyurethane tube after deployment. It can be seen from FIGS. 9a and 9b that the PLGA conformal coating transferred intact to the tube with the Sharpie™ ink markings.

For comparison, a balloon was prepared identically except that no PVP layer was provided. The balloon was deployed in the tube in water as described above. The PLGA coating remained adhered to the balloon when inflated. None of the film was transferred to the polyurethane tube.

### Example 2

### Crosslinked Alginate Carrier Film with Paclitaxel Drug Layer

A 3% solids solution of sodium alginate polysaccharide in water was syringe coated onto 3 mm×15 mm Liberte® balloons at a range of coat thicknesses (5 to 25 μl). While the coating was still wet the balloons were immersed in a 5% solution of calcium chloride for about 1 minute—this physically crosslinks the coating. The balloons were rinsed with DI water and dried. The alginate coated balloon was then syringe coated with 4% paclitaxel solution in acetone. The drug density was about 2 μg/mm². The coatings were dried and the balloons folded. The balloons were deployed in a polyurethane tube as described in Example 1.

FIG. 10a shows an image of the polyurethane tube after deployment of one of these balloons. The drug (white) is visible at high density. FIG. 10b shows an image of the same balloon after deployment. There is no visible drug on the balloon after deployment. Thus it appears that substantially all of the drug on the alginate coating was transferred from the balloon.

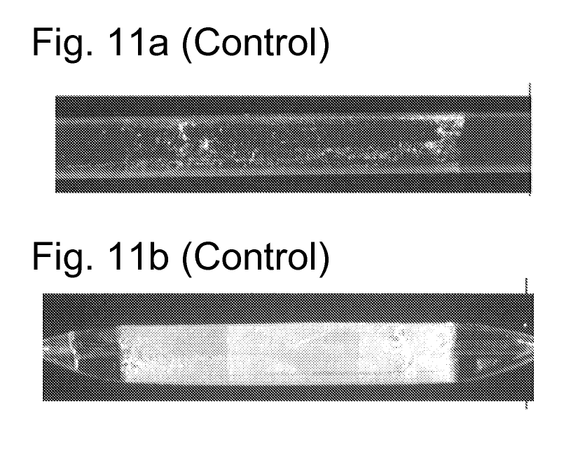

A control balloon with a coating of paclitaxel only (2 μg/mm²) was also coated on the same type of balloon but this time with no alginate layer and inflated in a polyurethane tube. FIG. 11a shows an image of the tube after deployment. Very little drug is visible on the tube after deployment. FIG. 11b shows the balloon after deployment. It can be seen that the vast majority of the drug is left on the balloon. Thus transfer efficiency is very low.

The above examples and disclosure are intended to be illustrative and not exhaustive. These examples and description will suggest many variations and alternatives to one of ordinary skill in this art. All these alternatives and variations are intended to be included within the scope of the claims, where the term “comprising” means “including, but not limited to”. Those familiar with the art may recognize other equivalents to the specific embodiments described herein which equivalents are also intended to be encompassed by the claims. Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all claims which possess all antecedents referenced in such dependent claim if such multiple dependent format is an accepted format within the jurisdiction. In jurisdictions where multiple dependent claim formats are restricted, the following dependent claims should each be also taken as alternatively written in each singly dependent claim format which creates a dependency from an antecedent-possessing claim other than the specific claim listed in such dependent claim.

1. A drug delivery balloon comprising:
a balloon wall, and
a carrier film of biodegradable polymer that
includes a drug therein, or
has a drug carried thereon, and
said carrier film is sufficiently non-adherent to the balloon wall that it can separate substantially entirely from the balloon wall when the balloon is expanded at a delivery site and remain in place at the tissue site.

2. A drug delivery balloon as in claim 1 wherein said drug is provided as a separate layer on the carrier film.

3. A drug delivery balloon as in claim 1 wherein said drug is included in said carrier film.

4. A drug delivery balloon as in claim 1 comprising both a drug in said carrier film and a drug layer on said carrier film.

5. A drug delivery balloon as in claim 1 wherein the drug comprises a lipophilic substantially water insoluble drug.

6. A drug delivery balloon as in claim 5, wherein the drug comprises one or more of paclitaxel, rapamycin, everolimus, zotarolimus, biolimus A9, dexamethasone, or tranilast.

7. A drug delivery balloon as in claim 1 wherein said drug comprises paclitaxel, at least a portion of which is in the form of paclitaxel dihydrate.

8. A drug delivery balloon as in claim 1 further comprising a release layer between the balloon wall and the carrier film.

9. A drug delivery balloon as in claim 1 wherein the carrier film has no adhesion to the balloon.

10. A drug delivery balloon as in claim 1 wherein the carrier film comprises a lactate polymer or copolymer.

11. A drug delivery balloon as in claim 1 wherein the carrier film comprises PLGA.

12. A drug delivery balloon as in claim 1 wherein the carrier film comprises a biodegradable crosslinked polymer.

13. A drug delivery balloon as in claim 1 wherein the carrier film comprises a biodegradable ionically crosslinked polymer.

14. A drug delivery balloon as in claim 13 wherein the biodegradable ionically crosslinked polymer is an acid functional polysaccharide.

15. A drug delivery balloon as in claim 14 wherein the acid functional polysaccharide is crosslinked with a biocompatible polyvalent cation selected from the group consisting of calcium, magnesium or iron.

16. A drug delivery balloon as in claim 13 wherein the acid functional polysaccharide comprises at least one of alginates, glycosaminoglycans, xanthan gum, carrageenan, tragacanth, gellan gum and pectins

17. A drug delivery balloon as in claim 17 wherein said carrier film is formulated to degrade in the body within 24 hours after delivery.

18. A method of delivering a drug to a treatment site in the body comprising
providing a medical device comprising a balloon as in claim 1,
advancing the balloon to the treatment site,
inflating the balloon,
deflating the balloon to separate said carrier film with said drug from the balloon wall, and
withdrawing the balloon leaving the carrier film with said drug in place at the treatment site.

19. A process for producing a medical device balloon comprising the steps of
providing a balloon having a balloon wall,
applying an layer of an ionically crosslinkable polymer to the balloon wall,
ionically crosslinking the ionically crosslinkable polymer layer, and
applying a drug to the ionically crosslinked polymer layer.

20. A process as in claim 19 further comprising the steps of
applying an layer of an extractable material to the balloon wall, before applying the ionically crosslinkable polymer, and
extracting the extractable layer after the ionically crosslinkable polymer has been applied.
